(19) United States
(12) Patent Application Publication
(10) Pub. No.: US 2004/0038226 A1
(43) Pub. Date: Feb. 26, 2004

(54) Transgenic animal model by mineralocorticoid receptor antisense expression

(76) Inventors: Frederic Jaisser, Malakoff (FR); Ahmed Beggaht, Paris (FR); Nicolette Farman, Paris (FR); Stefania Puttini, Paris (FR)

Correspondence Address: LARSON & TAYLOR, PLC, 1199 NORTH FAIRFAX STREET, SUITE 900, ALEXANDRIA, VA, 22314, US

(21) Appl. No.: 10/275,746

(22) PCT Filed: May 9, 2001
(86) PCT No.: PCT/FR01/01400

(30) Foreign Application Priority Data
May 9, 2000 (FR) 00/05885

(51) Int. Cl. C07K 14/72; C12Q 1/68; C07H 21/04; C12P 21/02; C12N 5/06

(52) U.S. Cl. 435/6; 435/69.1; 435/320.1; 435/325; 530/350; 536/23.5

(57) ABSTRACT

The invention concerns a nucleic acid construct blocking the expression of the murine mineralocorticoid receptor, in particular a vector containing an RNA antisense, and a non-human transgenic animal in whose genome said construct is incorporated, said animal being useful in particular as model for studying pathologies wherein the mineralocorticoid is involved.

The present invention relates to a nucleic acid construct which blocks the expression of the murine mineralocorticoid receptor, and to a transgenic animal in the genome of which this construct is integrated.

The receptor of mineralocorticoid hormones, also known as the mineralocorticoid receptor (MR) is expressed in wide variety of tissues, namely in particular in the epithelial cells which transport sodium, or in the non-epithelial cells of the cardiovascular system and of the central nervous system. This receptor, cloned in the human (WO 88/03168) and in the rat, is activated by aldosterone. Under the effect of its ligand, the receptor undergoes a translocation in the cell nucleus where it acts as a transcription factor after interaction with GRE (“glucocorticoid response element”) consensus sequences. The molecular mechanisms underlying the signalling route of the mineralocorticoids are not fully identified. Animal models are necessary in order to understand these mechanisms and would be particularly interesting especially as tools for research of medicaments acting on the pathologies in which the MR receptor is implicated.

From this viewpoint, transgenic mice which overexpress the human MR receptor in a constitutional manner have been constructed (Le Menuet et al, 2000), these mice showing renal lesions and cardiac functional alterations. The overexpression of the receptor in this model is generalised for all of the organs tested, in spite of the use of a promoter which should be specific, which complicates the study of the relationships between the receptor and the functions of each of the organs. Conversely, “knock-out” mice in which the murine gene of the MR receptor is invalidated have been obtained (Berger et al, 1998), but this invalidation involves premature mortality (10 days after birth) due to renal deficiency.

Therefore there was a need for a non-human animal model showing phenotypic alterations close to those observed in human pathologies in which the MR receptor is implicated.

The authors of the present invention have succeeded in obtaining transgenic animals which meet this need by integrating in their genome a nucleic acid construct which blocks the expression of the mineralocorticoid receptor.

By blocking the expression of the MR receptor specifically in the heart, they succeeded more particularly in developing a murine model of cardiac fibrosis accompanied by a cardiopathy. The results obtained are all the more surprising since the data reported hitherto, in the cases of activation of the receptor in the rat or the mouse, tended towards a contrary teaching: rats treated by aldosterone, agonist of the MR receptor, developed a perivascular cardiac fibrosis (Robert et al, 1995), whilst mice overexpressing the receptor suffered from cardiac deficiencies (Le Menuet et al, 2000).

The invention therefore relates to a nucleic acid, such as an antisense RNA, which blocks the expression of the MR receptor, preferably the murine MR receptor. This murine receptor, cloned by the authors of the present invention, comprises the nucleotide sequence SEQ ID no. 1 and/or SEQ ID no. 2. The sequence SEQ ID no. 1 codes for the N-terminal part of the MR receptor, the part which shows a significant divergence with the other receptors for glucocorticoids. The sequence SEQ ID no. 2 also corresponds to a fragment of the 5′ part of cDNA of the murine MR receptor.

The blocking of the expression of the MR receptor is specific, that is to say that it does not affect the expression of the other receptors for glucocorticoids.

The nucleic acid which blocks the expression of the MR receptor preferably comprises a sequence of approximately 50 to 2000 bases complementary to a sequence established from the nucleotide no. 1 of the sequence SEQ ID no. 1. Preferably, it comprises, or is constituted by, the complementary sequence of 316 base pairs (bp) of the sequence SEQ ID no. 1. It can also comprise the complementary sequence of the sequence SEQ ID no. 2.

“Nucleic acid which blocks the expression of the MR receptor” is also understood to mean a nucleic acid which comprises a nucleotide sequence which differs from the complementary sequence of the native sequence of the endogenous MR receptor by a reduced number of modifications of nucleotides, for example by a substitution of one or several bases or by a chemical modification of one or several bases, or a coupling with a marker molecule such as a fluorescent molecule.

Also included in the invention are, on the one hand, the nucleic acids which inhibit the transcription by forming triple helixes with the DNA or, on the other hand, the chimeric RNA-DNA oligonucleotides.

The invention also relates to a nucleic acid construct, such as a plasmidic vector, comprising said nucleic acid sequence which blocks the expression of the MR receptor, hereafter also designated the “antisense sequence”, in association with elements permitting the expression of this antisense sequence.

“Nucleic acid construct” is understood in particular to mean a nucleic acid such as DNA or RNA, linear or circular. Amongst the said elements permitting the expression of the antisense sequence are to be found a promoter of the transcription and possibly one or several transcription termination sequences.

“Transcription termination sequence” is understood to mean any sequence which permits stopping of the transcription, particularly a STOP site contained in a polyadenylation (polyA) sequence. This may be a polyA derived from a virus, in particular the polyA of the “Simian Virus 40” (SV 40) or a polyA deriving from a eukaryotic gene, in particular the polyA of the gene coding for phosphoglycerate kinase (pgk-1) or the polyA of the gene coding for rabbit β globin.

The nucleic acid construct according to the invention can also comprise at least one selection sequence.

“Selection sequence” is understood to mean a sequence which permits selection between the cells which have integrated the nucleic acid construct according to the invention and those in which the transfection has failed.

These selection sequences may be “positive” or “negative” and dominant or recessive. A “positive” selection sequence refers to a gene coding for a product which permits only the cells which carry this gene to survive and/or to multiply under certain conditions. Amongst these “positive” selection sequences may be mentioned in particular the sequences of genes for resistance to an antibiotic, such as for example neomycin (neo'), hygromycin, puromycin, zeoycin, blasticidine or phleomycin. Another possible selection sequence is hypoxanthine phosphoribosyl transferase (HPRT). The cells which carry the HPRT gene can grow on a HAT medium (containing aminopterine, hypoxanthine and thymidine), whilst the HPRT-negative cells die on the HAT medium.

Conversely, a “negative” selection sequence refers to a gene coding for a product which can be induced in order to kill in a selective manner the cells which carry the gene. Non-limiting examples of this type of selection sequences include the thymidine kinase of the herpes simplex virus (HSB-tk) and HRPT. The cells which carry the HSV-tk gene are killed in the presence of gancyclovir or of FIAU(1,(1,2-deoxy-2-fluoro-β-D-rabinofuranosyl)-5-iodouracil). The cells which carry the HPRT gene can be killed selectively by 6-thioguanine (6-TG).

Other examples of “positive” or “negative” selection sequences are well known to the person skilled in the art.

In an advantageous manner, the nucleic acid construct according to the invention can also comprise a detection sequence.

“Detection sequence” is understood to mean a sequence coding for a detectable protein, useful as a marker for easy evaluation of the level of expression of the protein in question. In this case reference is also made to a “reporter gene”. It may for example be a sequence coding for an enzyme such as β-galactosidase (β-GAL), alcohol dehydrogenase (ADH), alkaline phosphatase such as human Alkaline Phosphatase (APh), green fluorescent protein (GFP) and chloramphenicol acetyltransferase (CAT), luciferase, or any other detectable marker well known to the person skilled in the art.

The nucleic acid construct according to the invention can also comprise a sequence ISCE 1. This sequence of 18 base pairs is a sequence which is not present naturally in the genome of mammals. It corresponds to the site of recognition of the meganuclease of yeast I-Sce1 (Choulika et al, 1994; Cohen-Tannoudji et al, 1998, U.S. Pat. No. 5,830,729). It can be used to permit a targeted modification of the locus in which it is inserted (here in the transgene integrated in a stable manner in the genome of the transgenic animal or of derived cells). In the present case, this sequence can permit modification of the transgene and thus expression of another nucleotide sequence under the control of the inducible promoter or of another promoter.

According to a preferred embodiment of the invention, this construct comprises one or several elements which render the expression of the antisense sequence inducible or conditional.

These may in particular be elements known as “acceptors” of the system of induction by tetracycline. For that, the antisense sequence may be associated with an operating sequence of the system of resistance to tetracycline of the bacterial transposon Tn10.

In the bacteria containing this transposon, in the absence of tetracycline, the protein TetR bonds in the form of a dimer to operating sequences situated in the tetracycline operon, thus blocking the transcription of the gene coding for the protein of resistance to tetracycline. In the presence of the antibiotic, the tetR can no longer bond to the operating sequences; the inhibiting effect on the transcription of the resistance gene is then abolished. In 1992, Gossen and Bujard carried out the fusion of the proteins TetR and VP16 (protein of the virus Herpes Simplex Virus essential to the transcription of the premature viral genes). In genetically modified eukaryotic cells the resulting chimeric protein (tTA: “Tetracycline controlled TransActivator”) is capable of activating the expression of reporter genes situated downstream of tetO operating sequences. In the presence of tetracycline the chimeric protein tTA does not bond to tetO and does not activate the transcription of its target genes. The rtTA (reverse tTA) system can also be used. This is a mutant of tTA which is not active in a constitutional manner but necessitates the presence of tetracycline or of its derivatives (such as doxycycline) in order to transactivate the minimal tetO promoter (Furth et al, 1994; Gossen and Bujard, 1992; Gossen et al, 1995; Kistner et al, 1998).

The tTA or rtTA unit can be carried by the same construct or by another vector.

Nevertheless it is preferred to use two nucleic acid constructions, one carrying the antisense and the other carrying the elements, such as tTA, which permit the inhibition of the expression of the antisense in the presence of tetracycline.

In an advantageous manner the nucleic acid construct according to the invention which carries the antisense comprises, upstream and downstream:

a transcription termination sequence as defined previously;

an antisense sequence as defined previously;

a detection sequence as defined previously;

a transcription termination sequence as defined previously;

a bidirectional minimal promoter as described in Baron et al (1995), fused to a tetO operating sequence and being inserted between the antisense sequence and the detection sequence in order to permit the transcription of the antisense sequence upstream and of the detection sequence downstream.

Other systems which render the expression of the antisense sequence inducible or conditional can be used. The nucleic acid construct according to the invention can in particular comprise elements known as “acceptors” recognised by elements known as “inductors” supplied by another nucleic acid construct, the unit of “acceptor” and “inductor” elements permitting the regulation of the antisense expression.

The crossing of an “inductor” animal and an “acceptor” animal can be effected in an advantageous manner. In this case the expression of the antisense integrated in the form of a transgene in the acceptor animal will depend upon the functional expression of the inductor.

Mention may be made in particular, as examples of elements permitting the regulation of the antisense expression, of the GAL4 system (Wang et al, 1997), the ecdysone system (No D., 1996), or a system of recombinases. Use may be made for example of the Cre recombinase of the P1 bacteriophage (Abremski et al, 1983), or the FIp recombinase of yeast (Logie et al, 1995).

In the case of Cre recombinase (Metzger and Feil, 1999), the antisense sequence can be placed under the control of a strong or tissue-specific promoter. However, the transcription is blocked by the presence of a transcription termination site such as a polyadenylation site flanked by two LoxP sites (Cre recombinase recognition sites). One of the LoxP sites is situated between the promoter and the antisense sequence. If the Cre recombinase is not present, or not expressed, or even not active, the antisense transcription cannot take place. In the opposite case, the antisense is expressed.

The expression of the Cre Recombinase can itself be dependent upon a strong constitutional promoter, inducible and/or tissue-specific. The expression can also be secondary to a local or general infection by an adenovirus or a recombinant retrovirus.

These recombinases can be modified or bonded in an operating manner (in particular by fusion) to a sequence which provides them with a property of induction by an exogenous agent. Thus it is possible to use a recombinase fused to the domain for fixing of the receptor for oestrogens (ER) which has previously been mutated so that it no longer fixes the endogenous oestrogens. On the other hand, it can be activated by tamoxifen or by one of its analogues (Metzger et al, 1995). It is also possible to use a recombinase fused to other domains such as the domain for fixing of the ligand of the receptor for progesterone (PR) (Kellendonk et al, 1999) or the domain for fixing of the ligand of the receptor for glucocorticoids (GR) (Brocard et al, 1998). These domains are previously mutated so that they are no longer activated by their ligand but solely by synthetic molecules such as dexamethasone or RU486. Use may be made for example of the sequence Cre ER^{T2} (Indra et al, 1999) which is a sequence coding for a protein of which the recombinase activity is very easily inducible by tamoxifen or by its analogues.

The invention also relates to a host cell into which at least one nucleic acid construct as described previously has been transferred in a stable manner.

The term “host cell” comprises any mammalian cell or other eukaryotic cell, in culture or in vivo, as part of an organism, the said cell being capable of being previously fused or genetically modified.

It is also possible to transfer into the host cell a nucleic acid construct carrying an antisense sequence as defined previously and at least one nucleic acid construct comprising “inductor” elements permitting the regulation of the antisense expression in such a way that these two constructs are cointegrated into the genome of the said cell.

The transfer of the vector into the host cell can be carried out by means of standard techniques known to the person skilled in the art, for example by electroporation, precipitation with calcium phosphate (Sambrook et al, 1989) or lipofection.

In general terms, the nucleotide vectors according to the invention can be released in the naked form, that is to say exempt from any agent which facilitates the transfection or also in association with such an agent, which may for example be a chemical agent which modifies the cell permeability (such as bupivacaine), liposomes, cationic lipids or microparticles for example of gold, silica or tungsten.

The chosen mode of transfer depends principally upon the host cell, as is well known to the person skilled in the art.

More particularly, the present invention relates to the case where the host cell is an ovocyte, preferably from a mouse.

The nucleic acid constructs according to the invention can then be transferred into the fertilised ovocyte by microinjection techniques such as those described in Hogan et al, 1994, or by modification of genes in embryonal cells or also by nuclear transfer with the nucleus of genetically modified cells.

The present invention also relates to transgenic animals in which at least one nucleic acid construct according to the invention is integrated in the genome. Of course, these animals are non-human animals, preferably mammals, and more particularly rodents such as mice, rats or guinea-pigs, rabbits, cattle, pigs or sheep. It relates more particularly to mice. These animals can for example be of generation F0, or can preferably belong the F1 lines.

According to a preferred embodiment of the invention, an animal known as an “acceptor” is produced which carries a nucleic acid construct comprising an antisense sequence of which the expression is blocked and one or several “acceptor” elements of a system for regulation of the expression.

This “single transgenic” animal can then be crossed with a so-called “inductor” animal carrying a nucleic acid construct including so-called “inductor” elements permitting the regulation of the expression of the antisense sequence. The animal, known as a “double transgenic” animal, obtained in this way carried the two types of constructs and expresses the antisense sequence in a manner which is regulated by the said elements permitting the regulation of the expression of the antisense sequence.

Therefore the invention relates more particularly to a method of obtaining a “double transgenic” animal wherein the expression of the mineralocorticoid receptor is suppressed in an inducible or conditional manner, a method in which a “single transgenic” animal, in which a nucleic acid construct comprising a nucleic acid as defined previously and of which the sequence is designated as an “antisense sequence” has been integrated into the genome, is crossed with a transgenic animal in which a nucleic acid construct carrying elements permitting the regulation of the expression of the said antisense sequence in an inducible or conditional manner is integrated into the genome.

Alternatively, a “double transgenic” animal can be obtained directly by cointegration of the two types of nucleic acid constructs into an ovocyte or a stem cell of this animal.

In accordance with the present invention it is also possible to produce non-human animals by integration of a nucleic acid construct carrying not only the antisense sequence but also the set of elements for regulation of the expression thereof.

In an advantageous manner, the said elements for regulation of the expression of the antisense sequence are specific to a tissue of the animal. For this they can comprise a tissue-specific promoter which permits targeted expression of the antisense in certain tissues, for example the heart.

The invention also relates to the use of transgenic animals such as are described above for the screening of therapeutic agents effective in the prevention and/or the treatment of human or animal disorders or pathologies in which the mineralocorticoid receptor is implicated.

Thus the expression of the antisense sequence in the cardiac cells of transgenic mice led to the development of a cardiac fibrosis accompanied by a cardiopathy. This model shows phenotypic alterations close to those observed in several human cardiac pathologies.

The suppression of the expression of the antisense sequence in the cardiac cells of transgenic mice which have developed the pathological phenotype and in which the expression of the transgene is inducible or conditional leads in an advantageous manner to a disappearance of the cardiac fibrosis and of the cardiopathy.

Therefore these transgenic animals are particularly useful in order to understand the development and the consequences of cardiovascular diseases of this type and in order to develop new therapeutic strategies including the conventional strategy by means of pharmacological substances, and the strategy by gene therapy.

Therefore the invention relates more particularly to a method of screening agents which are active against cardiovascular diseases, such as cardiac fibrosis and/or cardiac insufficiency, in which the effect of the said agents on the appearance and the development of cardiac alterations in transgenic animals such as are described above is tested.

Amongst the compounds to be screened, particular mention may be made of the therapeutic classes involving the antagonists or agonists of steroidal receptors (mineralocorticoid hormones, androgens, glucocorticoids, oestrogens, progesterones), the agents which alter the signalling route of angiotensin (angiotensin receptor 1 and 2), agents inhibiting the angiotensinogen conversion enzyme, or also agents which occur on the signalling path of nitrogen monoxide (for example endotheline and its receptor).

In the same way, transgenic animals which express the anti-receptor MR antisense sequence in the central nervous system are useful for the study of the memory, mood disorders such as anxiety, depression, euphoria. For example, a mouse CamKII-tTA such as is described by Mansuy et al (1998a), Mansuy et al (1998b), Mayford et al (1996), permitting the conditional expression of different transgenes in the hippocampus, can be crossed with a single transgenic mouse according to the invention carrying an anti-receptor MR antisense sequence in association with the operator tetO. The mice obtained express the anti-receptor MR antisense specifically in the hippocampus.

Transgenic animals which express the antisense sequence in the renal cells are also useful for the study of the control of transport of salts, and for the study of arterial tension.

Moreover, transgenic animals which express the antisense sequence at the level of the skin are interesting models for the study of the secretion of ions which is observed for example in children suffering from mucoviscisdosis, as well as for the study of disorders of the differentiation of the cells of the skin.

An expression of the antisense sequence in the cells of the colon of a transgenic animal also provides a useful model for the study of cancers of the colon, and for the study of all the disorders of cell differentiation at this level.

The invention also relates to the study of the differential expression of genes or of proteins, for example in the course of the establishment or reversion of pathological phenotypes bonded to the mineralocorticoid receptor in transgenic animals such as are described above.

“Differential expression” is understood to mean any modification of the level of expression of a gene or of a protein within one and the same animal, over the course of time, or also any difference in the level of expression of a gene or of a protein between two animals showing a difference of phenotype, at a given instant. A modification of the level of expression of a gene or of a protein is generally judged to be significant when a decrease or an increase by a factor of 2 approximately, or more, is observed.

“Reversion of pathological phenotypes” denotes the return to a normal phenotype in animals which showed pathological characteristics.

These studies could advantageously be used by conventional methods such as DNA screening of chips (Schena et al, 1998) for the identification of target genes, or bidimensional electrophoresis for the identification of target proteins in proteomic studies (Dutt et al, 2000).

Cultures of cells obtained from these transgenic animals can be produced and put to use for example within the framework of a strategy of cell transplantation. They may be cultures of primary cells, obtained from the “single transgenic” or “double transgenic” animals described above. They may also be cells obtained by crossing these animals with other transgenic animals. For example, transgenic animals which permit an immortalisation of the cells in culture can be used. Stable cultures are then obtained. It is also possible to use “humanised” transgenic animals in order to obtain “humanised” cells.

The invention then also relates to a method of therapeutic treatment in which cultivated cells from these transgenic animals are implanted into a recipient organism requiring such a treatment.

The cells isolated from these transgenic animals can also serve as a cell model to replace animal experimentation. The toxicity of molecules with therapeutic potential can then be tested on these cells instead of testing them directly on the animal.

These isolated and possibly cultivated cells therefore also form part of the invention. The invention also relates to the use of these cells for the screening of therapeutic agents effective in the prevention and/or treatment of human and or animal disorders or pathologies in which the mineralocorticoid receptor is implicated.

The following drawings and examples illustrate the invention without limiting the scope thereof.

### LEGENDS OF THE DRAWINGS

FIG. 1 is a diagram of the nucleic acid constructs according to the invention.

FIG. 1A shows the diagram of construction of the transactivator.

FIG. 1B shows the diagram of construction of the vector used for the expression of the antisense of the murine MR receptor.

FIG. 2 is a diagram of the strategy for obtaining “double transgenic” mice.

FIG. 3A shows cross-sections of the heart of two lines of “double transgenic” mice αMHC-tTA/MR 9 and αMHC-tTA/MR 27, treated or not with doxycycline (Dox).

FIG. 3B is a RNase protection analysis (RPA) of ARN LacZ transcripts observed in the heart (H) and not in the kidney (K) of mice αMHC-tTA/MR 9 and αMHC-tTA/MR 27, treated or not with doxycycline.

FIG. 4A is a diagram showing that the expression of the antisense of the murine MR receptor in the heart of the mice αMHC-tTA/MR 27 and 9 is inhibited by doxycycline.

FIG. 4B is a diagram showing that the expression of the antisense induces a decrease in the amount of endogenous MR receptor in double transgenic mice by comparison with transgenic mice which express the transactivator alone.

FIG. 5 is a set of diagrams showing a comparison of the phenotypic effects observed in the “double transgenic” mice (αMHC-tTA/MR 27) relative to the single transgenic mice (αMHC-tTA).

FIG. 5A shows the increase in the heart weight/body weight ratio.

FIG. 5B shows the increase in the mass of the left ventricle.

FIG. 5C shows an increase in diameter of the cardiac left ventricle.

FIG. 5D shows a decrease in the parameter of cardiac ejection fraction. The values represent averages±standard deviations (3=n=6). The asterisks show the statistically significant difference between the single and double transgenic mice starting from the same group (p<0.05).

FIG. 6A shows a histological section of the heart of “double transgenic” mice (αMHC-tTA/MR 27, designated DT) compared with a section of the heart of “single transgenic” (αMHC-tTA, designated ST), the sections being coloured with Sirius red.

FIG. 6B is a diagram showing a significant difference between the quantity of collagen in the double transgenic mice relative to the single transgenic mouse. The values represent averages±standard deviations (3=n=6). The asterisks show the statistically significant difference between the single and double transgenic mice starting from the same group (p<0.05).

FIGS. 7A and 7B are graphs showing the effect of doxycycline on the body weight of male and female “double transgenic” mice (line MR 17).

FIG. 8 is a table showing the functional parameters measured by ultrasound cardiography in animals of wild or “double transgenic” genotype treated or not by doxycycline or treated by spironolactone.

FIGS. 9A and 9B are graphs showing the development of the body weight of “double transgenic” mice MR 17 relative to the controls.

FIG. 10 shows the mortality curve of male mice MR 17 relative to the control mice.

FIG. 11 shows the installation of the cardiac interstitial fibrosis in the “double transgenic” animals MR 27 and its control by treatment with doxycycline from birth for a period of 12 weeks.

FIG. 12 shows the effect of the treatment of “double transgenic” animals MR 17 by doxycycline, from birth and for a period of 12 weeks, on the development of the heart weight/body weight ratio.

FIG. 13 shows that the administration of doxycycline from birth in “double transgenic” animals MR 17 or MR 27 permits prevention of the death of these animals.

FIG. 14 shows the reversion of the pathological phenotype of “double transgenic” animals when these are treated with doxycycline.

FIG. 15 shows the effect of the administration of spironolactone (30 mg/kg/day) during the second and third months, in control or “double transgenic” animals not treated by doxycycline.

### EXAMPLES

### Example 1

Nucleotide Construct Carrying the Antisense

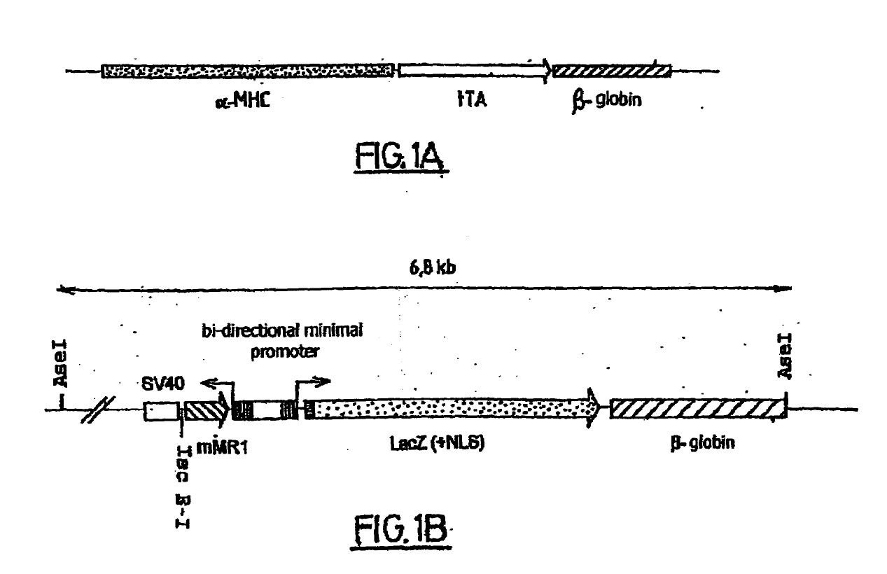

The nucleic acid construct prepared by the authors of the invention is shown schematically in FIG. 1B.

This is a plasmide vector pBI-3 (Clontech, reference 6150-1), modified as follows:

a) cloning of the cDNA coding for the N-terminal part of the mice mineralocorticoid receptor.

A reverse amplification has been carried out from total RNA extract from the brain of mice B6D2F1, using as primers olignonucleotides coding for two regions kept between the mineralocorticoid receptor sequence of rat and human.

| | |
|---|---|
| Sense primer: | |
| 5′ GGCTACCACAGTCTCCCTGAAGG 3′ | (SEQ ID no.3) |
| Antisense primer: | |
| 5′ CCATATATAAACCCATGGACTG 3′ | (SEQ ID no.4) |

After reverse transcription and amplification, a fragment of 316 pb (SEQ ID no. 1) is sub-cloned in the vector PGEM-T Easy (Promega, France). After sequencing, this fragment is excised from the cloning plasmid by enzymatic digestion PstI-notI. This fragment is then sub-cloned in antisense orientation (relative to the inducible promoter tet) in the vector Pbi-3 cut with the same restriction sites.

b) A restriction site for the meganuclease I-SceI is introduced with the aid of a dimer oligonucleotide into the SaII site situated between the antisense sequence of the MR of mice and the polyadenylation sequence SV40polyA. The insert is excised from the vector by digestion AseI, then purified by Elutip. The insert is quantified on agarose gel, diluted to the concentration of 4 ng/il in Tris 10 mM/EDTA 0.1 mM buffer and is ready to be microinjected.

### Example 2

Production of Transgenic Mouse

Lines of “single transgenic” mice were obtained in accordance with the following protocol:

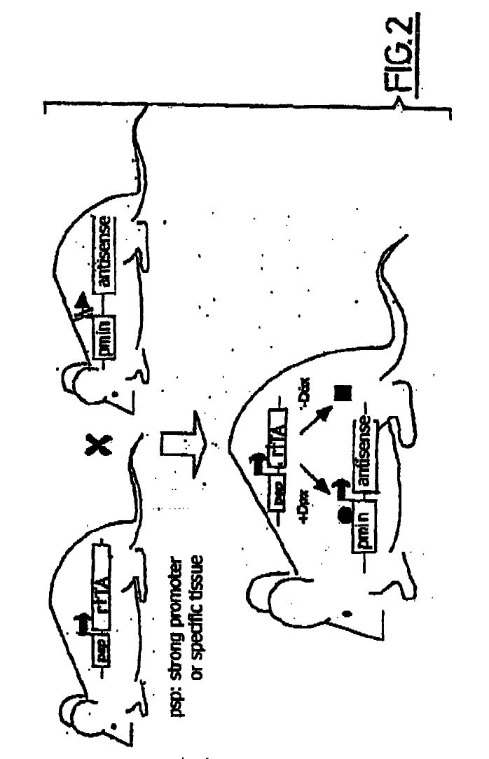

The insert is microinjected into the male pronucleus of fertilised ovocytes of mice B6D2F1 according to a standard procedure then reimplanted into a pseudogestant recipient mouse (Hogan et al, 1994). After birth, the animals are genotyped then the transgenic animals F0 (having integrated the insert in a stable manner) are identified and crossed in order to establish the lines. The transgenic animals of the first generation F1 are then crossed with αMHC-tTA mice sold by Jackson Laboratory, USA, under the reference number 003170 (FIG. 2). These αMHC-tTA mice are also called, by the Jackson Laboratory, FVB/N-TgN (MHCA tTA) 6 Smbf mice.

The specificity of this line for the cardiac myocytes is determined by a gene in which approximately 2.9 kb of the sequence 5′ flanking the gene of the heavy chain of myosine alpha of rat leads to the expression of the transactivator controlled by tetracycline (tTA, FIG. 1A), specifically in the cardiac myocytes (Yu et al, 1996).

The functional activity of the transactivator is modulated by the presence of an exogenous ligand which is not present in the natural state in mammals, doxycyline and its analogues. If the animals receive doxycycline (by intra-peritoneal injection, in drinking water or in food) the functional activity of tTA decreases. The expression of the secondary transgene is then progressively extinguished in the “double transgenic” mice, namely in the present case the mMR antisense. By placing the animals under doxycycline from gestation, the expression of the antisense is averted, which makes it possible to choose the moment when it is wished to set off the pathological process.

### Example 3

Inducible Expression of mMR Antisense RNA

a) Materials and Methods:

The molecular and phenotypic analyses were carried out on animals of 14 weeks. When necessary, doxycycline was administered for 8 days in the drinking water (0.4 mg/ml) and by peritoneal injection (2 mg/injection). For other experimental protocols, the mice receive doxycycline from the start of gestation in the mother.

Transcript Expression Study:

The molecular expression of the LacZ gene and of the antisense RNA was analysed by the RNase A protection technique with the aid of appropriate probes. The expression of the endogenous MR receptor was followed with the aid of oligonucleotide probes obtained from the sequence SEQ ID no. 1 or SEQ ID no. 2. SEQ ID no. 2 corresponds to a fragment of the cDNA, in its part 5′, of the total sequence of the endogenous MR receptor. This sequence was cloned by reverse amplification from total RNA extracted from the heart of mice B6D2F1, using as primers oligonucleotides which code for two highly conserved regions between the mineralocorticoid receptor sequence of rat and human.

| | |
|---|---|
| Oligonucleotide sense: | |
| 5′-AAGAGCCCTATCATCTGTCATGAGAA-3′ | (SEQ ID no.5) |
| Oligonucleotide antisense: | |
| 5′-GGACTGGAGACTGGAGATTTTACACTGC-3′ | (SEQ ID no.6) |

After reverse transcription and PCR amplification, a fragment of 320 bp(SEQ ID no. 2) is sub-cloned in the vector pT-Adv (Clontech, France). The PCR fragment is then sequenced and its orientation confirmed. The construct is used for the synthesis of an antisense probe used in RNase Protection Assay (RPA) for the detection of mMR transcripts.

Complementary Analyses:

The function expression of the LacZ gene is obtained by conventional X-Gal staining on a frozen section of heart. The histological analysis was carried out by Sirius red staining on a section of frozen heart. The analysis of the functional parameters was evaluated by cardiac ultrasound on anaesthetised animals.

b) Results

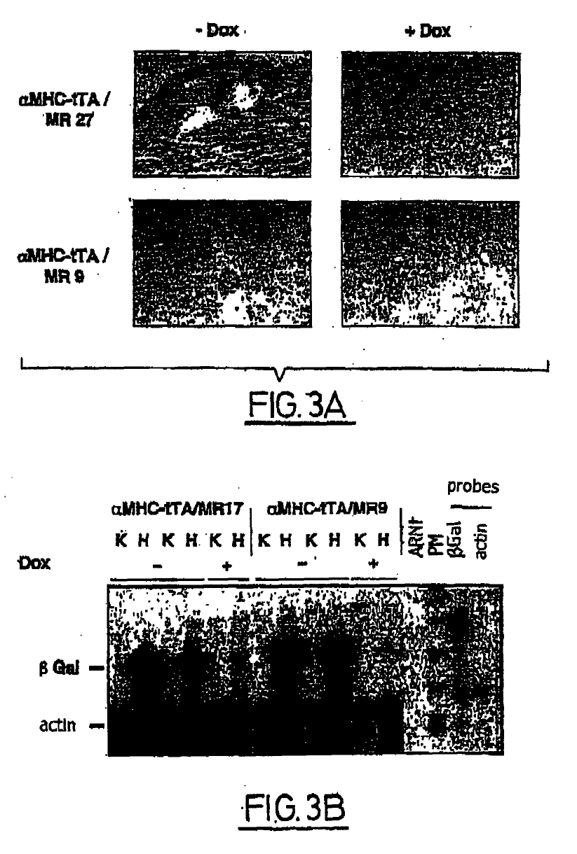

Mice of lines αMHC-tTA/MR 9 or 27 were treated or not with doxycycline (Dox) for eight days. The hearts were collected and fine sections were fixed in 4% paraformaldehyde then stained in a solution of X-Gal. For each of the lines MR 9 and MR 27 a nuclear LacZ staining was observed in all the cardiomyocytes in the absence of doxycycline, the expression being practically completely suppressed in the presence of doxycycline (FIG. 3A).

Moreover, the RNA of the liver and the heart of “double transgenic” mice MR 9 and MR 17 was extracted, the animals having been treated or not with doxycycline for eight days. The RNase protection method was carried out for each of these transgenic lines on three animals of which two were in the absence (−) of doxycycline and one in the presence (+) of doxycycline. In the heart (H) an expression of RNA is observed which is greatly reduced by the doxycycline. In the kidney (K) there is no expression in the presence or in the absence of doxycycline. The integrity of the RNA is controlled using an actin probe (FIG. 3B).

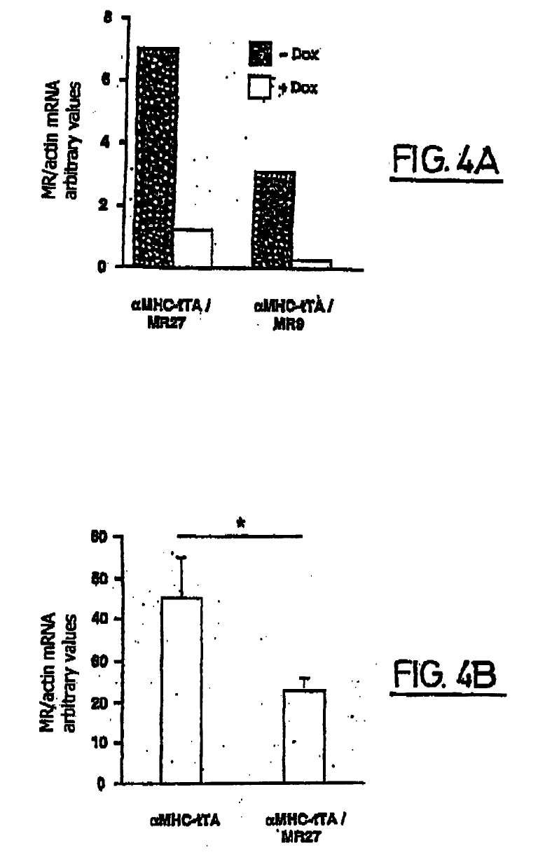

FIG. 4A shows by analysis of a RNase protection test that the expression of the mMR antisense is detected in these mice and that its expression can be regulated by doxycycline. FIG. 4B shows the measurement, in the stable state, of the expression of the RNA coding for the mineralocorticoid receptor. A decrease of approximately 50% of the residual expression of MR RNA is observed in “double transgenic” animals relative to the “single transgenic” control animals (MHC-tTA). This effect is present in the males and the females.

It should be noted that the basic expression is higher in the females and that the effect of the antisense RNA comes close to the level of expression of the endogenous mMR observed in the control males (FIG. 4B). This could be important in order to explain the phenotypic difference observed between the males and the females, these latter being less affected than the males.

### Example 4

Consequences on the Morphometry and the Cardiac Function

a) Onset of the Pathology Over the Course of Time

The “double transgenic” animals aged one month do not show any clear clinical or function signs nor any evident anatomo-pathological signs. On the other hand, from the second month the pathology is established.

b) Phenotype of the “Double Transgenic” Animals

Line MR 27

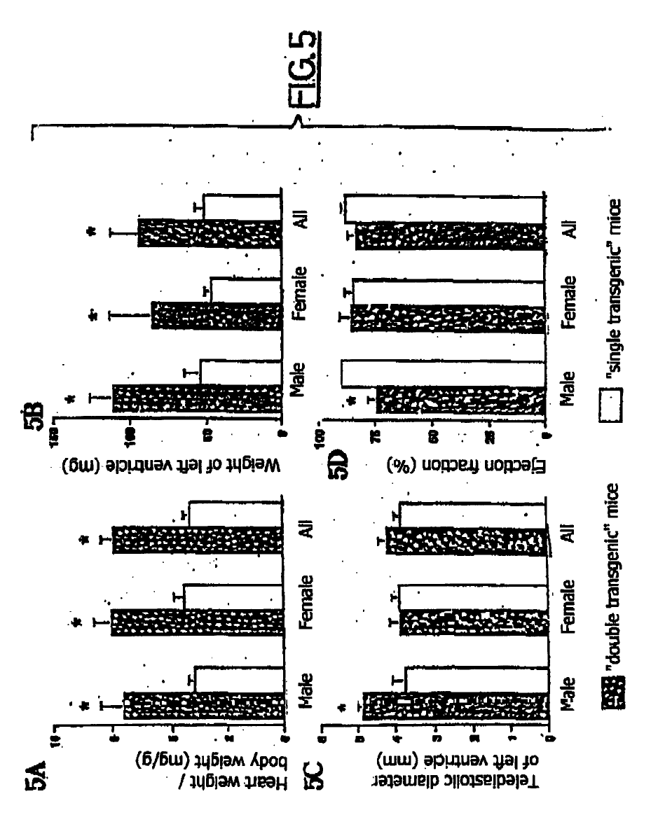

In the “double transgenic” animals which express the mMR antisense, a significant increase in the weight of the heart is observed by comparison with mono-transgenic control animals (FIG. 5A) aged 3.5 months. This is observed in the males and the females, with a great dispersion of measurements in the case of the latter. An increase in the bodyweight of “double transgenic” males is also observed relative to the male controls. This could reflect hydrosodium retention which may be associated with the onset of cardiac insufficiency.

From the haemodynamic point of view, the ultrasound measurements carried out on the animals one week before sacrifice reveal a significant decrease in the systolic ejection fraction in males (FIG. 5D), as well as parameters which evoke a dilated cardiopathy in the double transgenic animals, by comparison with the control animals (FIGS. 5B and 5C).

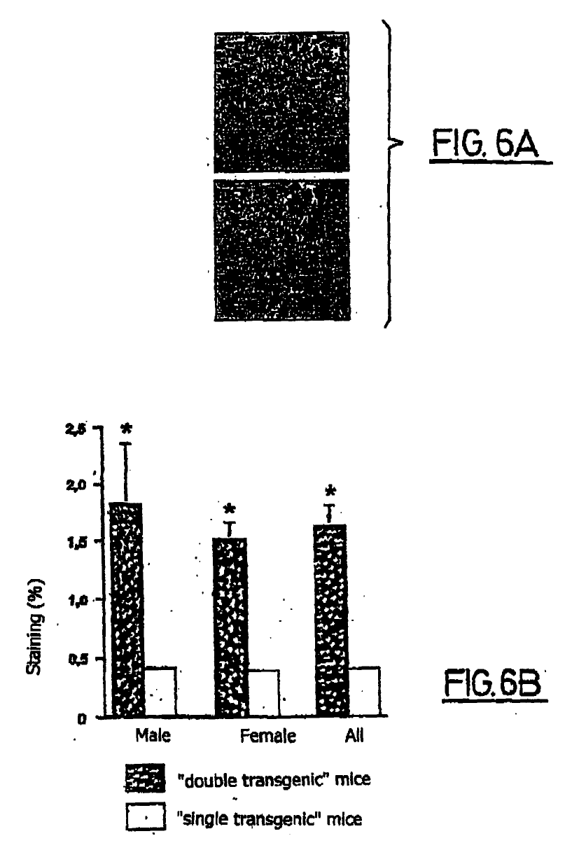

The histological analysis of the heart of “double transgenic” animals also reveals a substantial interstitial fibrosis of the two ventricles without perivascular fibrosis (FIGS. 6A and 6B). The interstitial collagen deposit proves to be significantly correlated with the residual quantity of messenger RNA of the endogenous mMR.

The plasmatic concentrations of aldosterone or of corticosterone in the “double transgenic” animals are comparable to those measure in the control mice. Therefore the phenotype observed is not associated with a modification of the mMR ligand level.

Line MR 17

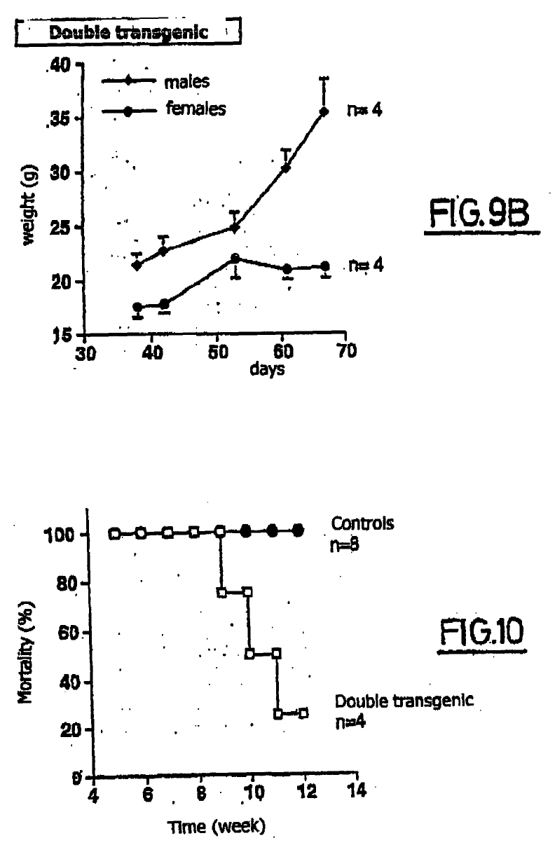

As observed previously with the line MR 27, the “double transgenic” animals of the line MR 17 show an increase in the body mass (FIGS. 9A and 9B) as well as a cardiac hypertrophy.

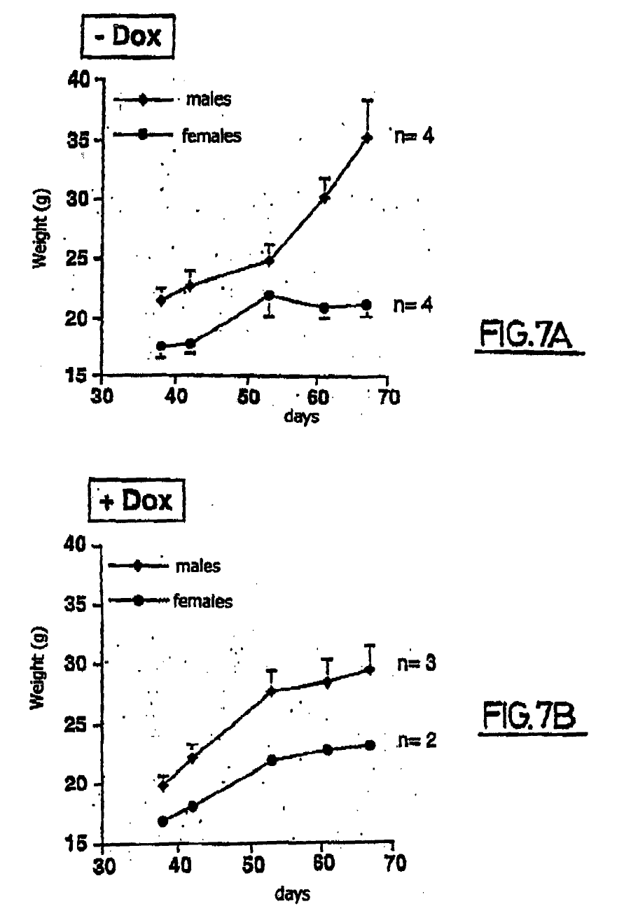

The line MR 17 shows a much more severe phenotype, leading to the death of approximately 75% of the male animals in less than three months (FIG. 10). These animals die from a major cardiac failure, attested by clinical signs (FIGS. 7A and 7B) and haemodynamic signs (cardiac ultrasound scan). By contrast, the mortality is reduced in females. The death of the male animals occurs between two and three months due to terminal cardiac insufficiency. The autopsy on the animals shows typical signs of advanced cardiac insufficiency (anasarca), cutaneous oedema, ascites, pleural effusion, hepatomegalia.

The above results indicate that the male mice are more severely afflicted than the female mice. Thus the majority of the males suffer from a severe cardiac insufficiency accompanied by clinical signs of anasarca (observed in human pathology), leading to death. These results suggest a participation of the androgenic receptor or of the androgenic hormones in the appearance of a more severe phenotype.

### Example 5

Monitoring of the Appearance of the Pathological Phenotype

a) Blockage of the Establishment of the Pathological Phenotype

The observations presented in Example 4 have made it possible to show that in each of the lines MR 27 and MR 17 the appearance of the phenotype can be averted by the inhibition of the expression of the mMR antisense RNA.

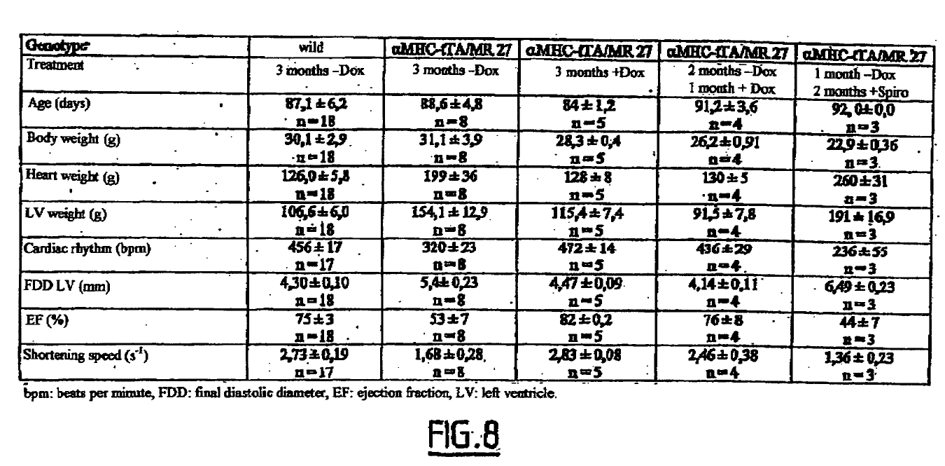

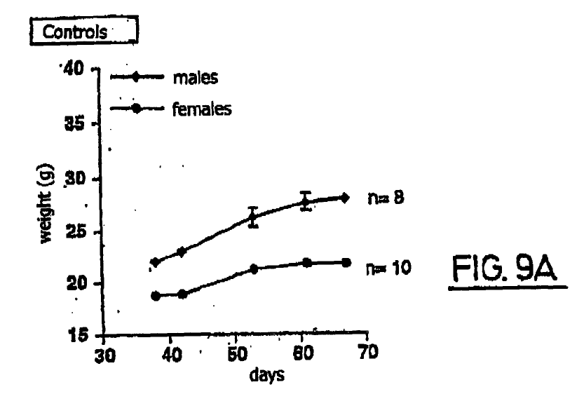

Thus the appearance of the interstitial fibrosis in the “double transgenic” animals of the line MR 27 can be prevented by the continuous administration of doxycycline from birth (FIG. 11). FIG. 8 shows moreover that the doxycycline makes it possible to restore the normal functional parameters in the “double transgenic” animals treated.

Equally, in the line MR 17 the administration of doxycycline continuously in drinking water from gestation in the mother then in the animals after their birth makes it possible to monitor and to avert the appearance of the drastic phenotype observed in the male “double transgenic” animals (FIG. 11).

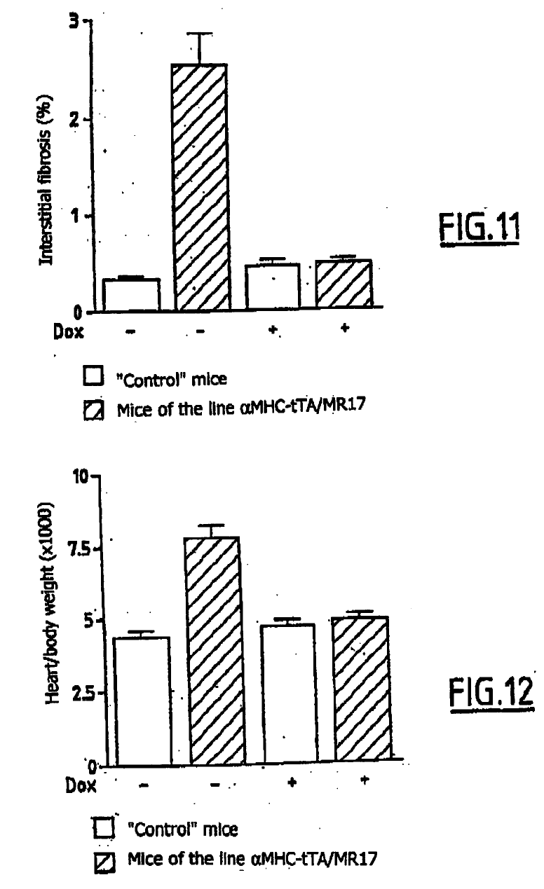

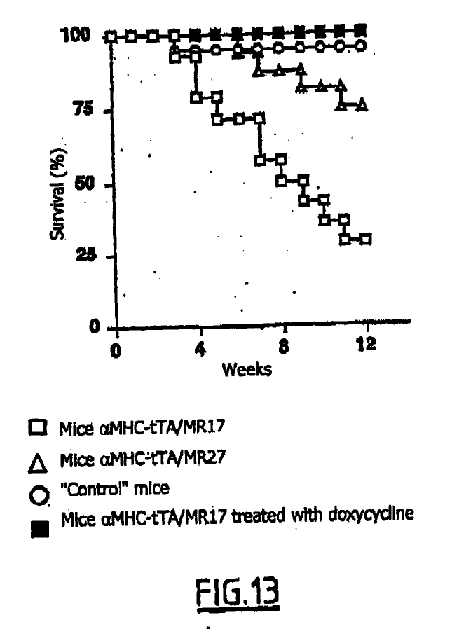

Thus the increase in the heart weight/body weight ratio is averted (FIG. 12). For each of the lines, the continuous administration of doxycycline from birth makes it possible to prevent the death of the “double transgenic” animals. These then show a survival curve comparable to that of the “wild” or monotransgenic control animals (FIG. 13).

b) Induction of the Pathological Phenotype in the “Double Transgenic” Adult Animal

The appearance of the pathology in the adult “double transgenic” animal (two months) which was previously treated with doxycycline can be induced by suppressing the administration of doxycycline.

Thus the use of a system of inducible and therefore conditional expression makes it possible to monitor the expression of the transgene over the course of time and therefore the establishment of the pathological phenotype.

Therefore the model which has been developed opens up interesting perspectives for the very precise study of the establishment of the cardiac phenotype whilst avoiding the phenomena of compensation which may occur over the course of the embryogenesis or in the post-natal period. Therefore this comes more faithfully close to the pathogenic conditions observed in human pathology.

### Example 6

Reversibility of the Pathological Phenotype

The use of the system of inducible tet expression also makes it possible to study the reversibility of the pathological phenotype when the expression of the mMR antisense RNA is suppressed.

The kinetics of disappearance of the pathological alterations was analysed in “double-transgenic” animals of the line MR 27 aged two months, in which the pathological phenotype is already established. These animals were treated in a continuous manner with doxycycline (at 2 mg/ml in drinking water) for one month so as to block the expression of the mMR antisense RNA.

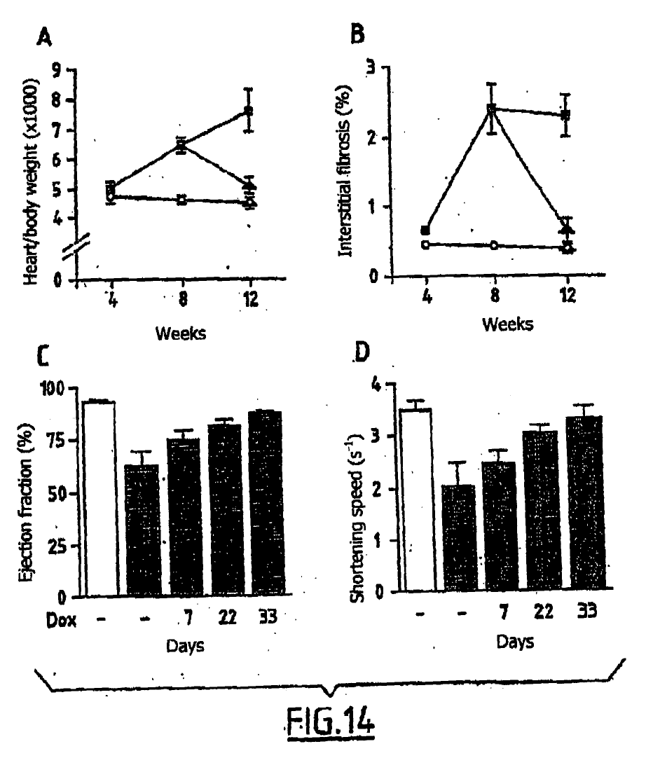

FIG. 5 shows a return to normal functional parameters, that is to say parameters comparable to those of the control animals, for animals of the line MR 27 treated with doxycycline. More particularly, the heart weight/body weight ratio (FIG. 14A) and the ultrasound cardiography parameters (FIGS. 14C and D) return to normal values.

The improvement in the ultrasound cardiography parameters can be detected from the first week after the start of treatment with doxycycline (FIGS. 14C and D).

The disappearance of the cardiac interstitial fibrosis (FIG. 14B) suggests that the abnormal deposit of extracellular matrix was reversible.

In fact, the suppression of the expression of mMR antisense RNA permits the reversion of the cardiac phenotype within the month following the start of treatment with doxycycline.

Only the use of a conditional, inducible and reversible system (such as the tetracycline system) makes it possible to tackle these questions. The model according to the invention is unique since it offers for the first time the possibility of analysing the mechanisms involved in the reversibility of a cardiac fibrosis without the use of a pharmaceutical drug.

### Example 7

Synergism of Spironolactone, an mMR Antagonist, and of the Expression of the mMR Antisense

Spironolactone is a classic inhibitor of mMR which is used as a diuretic in hypertension and in cardiac insufficiency. The effect of early treatment with spironolactone (30 mg/kg/day) was evaluated on “double transgenic” animals aged one month, not treated with doxycycline, an age at which the phenotype is still normal.

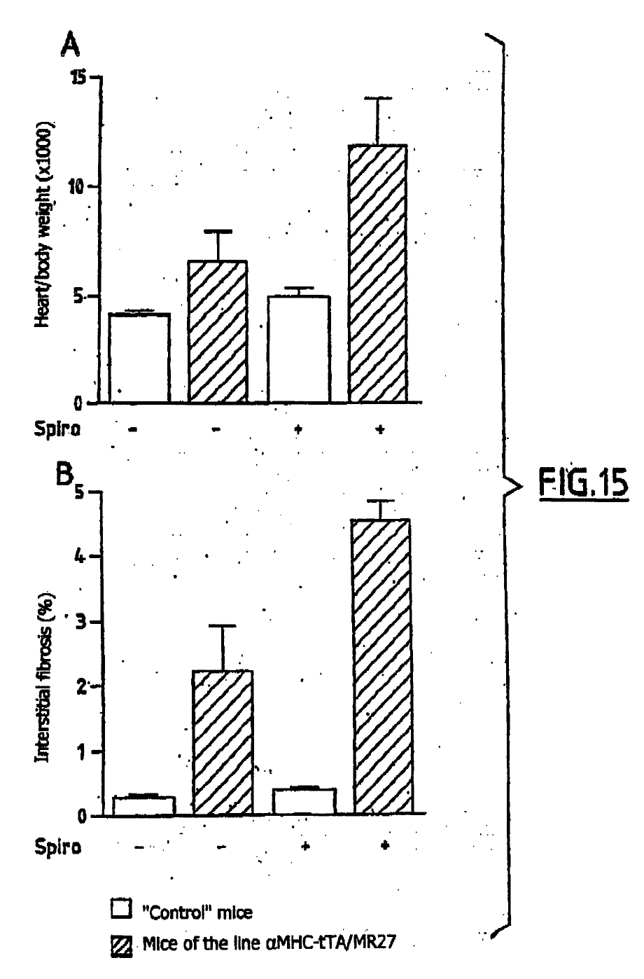

After two months of treatment, the “double transgenic” animals treated with spironolactone show an aggravation of the pathological phenotype by comparison with the untreated “double transgenic” animals. In particular a supplementary increase in the heart weight (FIG. 15A) and in the cardiac interstitial fibrosis (FIG. 15B) is observed. Moreover the functional ultrasound cardiography parameters are greatly altered (FIG. 8).

The administration of spironolactone to control animals (wild or single transgenic) does not produce any functional or structural alteration (FIGS. 15A and B).

Therefore the spironolactone has a synergistic effect with the expression of the mMR antisense RNA but does not, alone, permit the appearance of the pathological phenotype to be induced.

### Example 8

Differential Expression of Genes in “Control” or “Double Transgenic” Animals

The analysis of the differential expression of genes is carried out commercial glass plates (Atlas glass microarrays, Clontech) where more than 1000 different mouse genes are represented. The fluorescent probes used in order to analyse the level of expression of the genes are prepared from specimens of RNAs obtained from the hearts of transgenic animals with the aid of the “Atlas glass fluorescence labelling kit” (Clontech).

The expression ratio corresponds, for a given gene, to the ratio of the expression measured in an experimental situation (“double transgenic animals”) to the expression measured in a control situation (“control” animals). A difference of expression is generally judged to be significant when the ratio is higher than approximately 2, or lower than approximately 0.5.

The following table illustrates the differences of expression observed between “control” and “double transgenic” animals aged 1 month:

| | Ratio of |
|---|---|
| | expression |
| | experimental/ |
| | control in |
| | animals of 1 |
| Gene | month |
| GATA-binding protein 4 | 11,6 |
| transducin beta-2 subunit | 10,7 |
| T-cell leukemia, homeobox 1 | 9,5 |
| low density lipoprotein receptor | 8,3 |
| uracil-DNA glycosylase | 7,3 |
| radical fringe gene homolog | 7,1 |
| related to Drosophila groucho gene | 7,1 |
| wingless-related MMTV integration site 3 | 6,3 |
| insulin-like growth factor receptor II | 5,5 |
| ubiquitously-expressed nuclear receptor | 5,4 |
| dentin sialophosphoprotein | 5,2 |
| wingless-related MMTV integration site 7 | 5,1 |
| ribosomal protein S29 | 4,4 |
| interferon beta, fibroblast | 4,4 |
| genomic screened homeo box 2 | 4,2 |
| LIM homeo box protein 4 | 4,1 |
| GATA-binding protein 2 | 4,1 |
| myosin heavy chain | 4,0 |
| semaphorin E | 3,9 |
| Glyceraldehydes-3-phosphate dehydrogenase | 3,9 |
| aryl hydrocarbon receptor nuclear transl | 3,9 |
| chromobox homolog 4 (Drosophila) | 3,8 |
| activin receptor IIB | 3,7 |
| L+ | 3,7 |
| interleukin 15 | 3,7 |
| period homolog (Drosophila) | 3,7 |
| dlk1-like homolog (Drosophila) | 3,7 |
| HNF-3/forkhead homolog, brain factor 1 | 3,6 |
| myelin protein zero | 3,6 |
| hypoxanthine guanine phosphoribosyl transferase | 3,6 |
| Myeloblastosis oncogene-like 1 | 3,5 |
| POU domain, class 2, transcription factor | 3,4 |
| retinoic acid receptor, alpha | 3,4 |
| zinc finger protein 36 | 3,4 |
| defender against cell death 1 | 3,4 |
| transcription factor 21 | 3,4 |
| nitric oxide synthase 2, inducible, macrophage | 3,4 |
| LIM homeo box protein 3 | 3,3 |
| inhibin beta-B | 3,3 |
| ephrin A2 | 3,2 |
| basic Kruppel-like factor | 3,2 |
| Adrenomedullin receptor | 3,2 |
| alpha internexin neuronal intermediate filament | 3,2 |
| oncostain M | 3,2 |
| zinc finger protein 144 | 3,1 |
| prothymosin alpha | 3,1 |
| vascular endothelial growth factor | 3,1 |
| Friend leukemia integration 1 | 3,1 |
| Ubiquitin | 3,1 |
| linker for activation of T cells (LAT) Z | 3,1 |
| heat shock protein, 84 kDa 1 | 3,1 |
| Notch gene homolog 1, (Drosophila) | 3,0 |
| Cf2r; coagulation factor II (thrombin) r | 3,0 |
| immunoglobulin S mu binding protein 2 | 3,0 |
| lymphotoxin A | 3,0 |
| glial cell line derived neurotrophic fac | 3,0 |
| endothelin receptor type B | 3,0 |
| lunatic fringe gene homolog (Drosophila) | 3,0 |
| cordon-bleu | 3,0 |
| laminin, alpha 2 | 2,9 |
| protein phosphatase 1A, magnesium depend | 2,9 |
| L1 cell adhesion molecule | 2,9 |
| nur related protein 1 | 2,9 |
| guanine nucleotide binding protein, alpha | 2,9 |
| single-minded 2 | 2,8 |
| GLI-Kruppel family member GLI | 2,8 |
| homeobox protein 2.4 (Hox-2.4) | 2,8 |
| neurofibromatosis 1 | 2,8 |
| gap junction membrane channel protein beta | 2,8 |
| interleukin 1 receptor antagonist | 2,8 |
| homeo box A9 | 2,7 |
| cofilin 1, non-muscle | 2,7 |
| fibroblast growth factor 13 | 2,7 |
| glucose phosphate isomerase 1 complex | 2,7 |
| neuropeptide nociceptin 1 | 2,6 |
| ubiquitin-conjugating enzyme E2B | 2,6 |
| serine protease inhibitor 3 | 2,6 |
| transducer of ErbB-2 | 2,6 |
| kinesin family member 3a | 2,5 |
| Drosophila NK2 transcription factor | 2,5 |
| distal-less homeobox 2 | 2,5 |
| signal transducing adaptor molecule | 2,5 |
| MAP kinase-activated protein kinase 2 | 2,5 |
| gamma-aminobutyric acid (GABA-A) transporter | 2,5 |
| heat shock protein, 60 kDa | 2,5 |
| C5A receptor | 2,4 |
| distal-less homeobox 3 | 2,4 |
| T-cell lymphoma invasion and metastasis | 2,4 |
| homeo box B5 | 2,4 |
| eyes absent 1 homolog (Drosophila) | 2,4 |
| homeo box A1 | 2,4 |
| cystatin 3 | 2,4 |
| distal-less homeobox 5 | 2,4 |
| homeo box, msh-like 2 | 2,4 |
| H6 homeo box 3 | 2,4 |
| E74-like factor 1 | 2,4 |
| phenylethanolamine-N-methyltransferase | 2,4 |
| ERBB-3 receptor | 2,3 |
| endothelin 3 | 2,3 |
| tyrosine kinase receptor 1 | 2,3 |
| cytokine inducible SH2-containing proteine | 2,3 |
| ELK1, member of ETS oncogene family | 2,2 |
| CD7 antigen | 2,2 |
| glutamate receptor, ionotropic, kainate | 2,2 |
| nuclear factor of activated T-cells, cytokine | 2,2 |
| patched homolog 2 | 2,2 |
| spleen tyrosine kinase | 2,2 |
| interferon regulatory factor 2 | 2,1 |
| xeroderma pigmentosum, complementation | 2,1 |
| acetylcholine receptor delta | 2,1 |
| v-crk-associated tyrosine kinase substrate | 2,1 |
| raf-related oncogene | 2,1 |
| cyclin F | 2,1 |
| Tnf receptor-associated factor 3 | 2,1 |
| Eph receptor B2 | 2,1 |
| promyelocytic leukaemia | 2,1 |
| myelin-associated oligodendrocytic basic | 2,1 |
| tyrosine 3-monooxygenase/tryptophan 5-monooxygenase | 2,1 |
| atonal homolog 2 (Drosophila) | 2,0 |
| paired box gene 5 | 2,0 |
| AT motif binding factor 1 | 2,0 |
| excision repair 1 | 2,0 |
| retinoic acid receptor, beta | 2,0 |
| DNA-damage inducible transcript 3 | 2,0 |
| cell division cycle 25B | 2,0 |
| cell division cycle 2-like 1 | 1,9 |
| Erf; Ets-related transcription factor | 1,9 |
| Bcl2-associated athanogene 1 | 1,9 |
| SKELEMIN | 1,9 |
| membrane transporter protein | 1,9 |
| early lymphoid specific transcription factor | 1,9 |
| hormone receptor | 1,9 |
| laminin, beta 3 | 1,9 |
| myosin light chain, alkali, nonmuscle | 1,9 |
| guanylate kinase membrane-associated | 1,9 |
| lysosomal-associated protein transmembrane | 1,9 |
| homeo box A11 | 0,5 |
| Relaxin | 0,4 |
| MyoD family inhibitor | 0,3 |
| Vitronectin | 0,3 |
| lung Kruppel-like factor | 0,2 |
| homeo box D4 | 0,2 |

The same type of experiment was carried out directly by Clontech on animals of 3 months (Atlas mouse 2 array, Clontech). The results are set out in the following table:

| | Ratio of |
|---|---|
| | expression |
| | experimental/ |
| | control in |
| | animals of |
| Gene | 3 months |
| Alzheimer's disease amyloid A4 protein precursor | 10,0 |
| homologue | |
| Angiotensin-converting enzyme (ACE) | 6,7 |
| growth arrest & DNA-damage-inducible protein 153 | 6,5 |
| (GADD153) | |
| semaphorin B | 6,0 |
| fms-related tyrosine kinase 3 Flt3/Flk2 ligand | 6,0 |
| cytokine inducible SH2-containing protein 7 (CISH7) | 6,0 |
| protease nexin 1 (PN-1) | 6,0 |
| serine protease inhibitor 2-2 (SPI2-2) | 6,0 |
| retinoic acid-inducible E3 protein | 5,1 |
| interferon regulatory factor 1 (IRF1) | 5,0 |
| P-selectin glycoprotein ligand 1 precursor (PSGL1) | 5,0 |
| matrix metalloproteinase 2 (MMP2) | 5,0 |
| T-cell death-associated protein (TDAG51) | 4,8 |
| bone/cartilage proteoglycan I precursor (PGI) | 4,5 |
| calpactin I light chain | 4,2 |
| CD14 monocyte differentiation antigen precursor | 4,0 |
| non-muscle myosin light chain 3 (MLC3NM) | 3,6 |
| zyxin (ZYX) | 3,5 |
| non-muscle cofilin 1 (CFL1) | 3,3 |
| syndecan 3 (SYND3) | 3,1 |
| paired mesoderm homeobox protein 2 (PMX2; PRX2) | 3,0 |
| proliferation-associated protein 1 (PLFAP) | 3,0 |
| neural cadherin precursor (N-cadherin; CDH2) | 3,0 |
| fibronectin 1 precursor (FN1) | 3,0 |
| MCM5 DNA replication licensing factor (CDC46 | 3,0 |
| homolog) (P1-CDC46). | |
| RAD23 UV excision repair protein homolog B | 3,0 |
| (MHR23B; RAD23B) | |
| vimentin (VIM) | 2,9 |
| cytoplasmid dynein light chain 1 | 2,9 |
| bcl-2 homologous antagonist/killer (BAK1) | 2,6 |
| laminin gamma 1 subunit precursor (LAMC1) | 2,6 |
| CYSTATIN C PRECURSOR (CYSTATIN 3) | 2,6 |
| cadherin 5 (CDH5) | 2,5 |
| macrophage colony stimulating factor 1 (CSF-1) receptor | 2,5 |
| bone morphogenetic protein 1 precursor (BMP1) | 2,5 |
| oncostatin M (OSM) | 2,5 |
| cathepsin H | 2,5 |
| transforming growth factor beta 1 (TGF-beta 1; TGFB1) | 2,3 |
| apolipoprotein E precursor (apo-E) | 2,3 |
| cathepsin D (CTSD) | 2,2 |
| integrin-linked kinase (ILK) | 2,2 |
| lysosomal protective protein precursor (EC 3.4.16.5) | 2,2 |
| (cathepsin A) (carboxypeptidase C) (MO54). | |
| bone proteoglycan II precursor (PG-S2) (Decorin) | 2,2 |
| (PG40).(DCN) | |
| endoglin precursor (EDG; ENG); cell surface MJ7/18 | 1,9 |
| antigen | |
| DiGeorge syndrome chromosome region 6 protein | 0,6 |
| (DGCR6) | |
| related to Drosophila groucho gene (GRG) | 0,5 |
| LIM domain-binding protein 1 (LDB1) | 0,5 |
| laminin alpha 5 subunit precursor (LAMA5) | 0,5 |
| granulocyte-macrophage colony-stimulating factor | 0,5 |
| receptor low-affinity subunit precrusor (GM-CSF-R) | |
| Frataxin | 0,5 |
| transcription factor 15 (TCF15) | 0,5 |
| B-raf proto-oncogene | 0,5 |
| microsomal glutathione S-transferase (MGST1; GST12) | 0,4 |
| nucleoside diphosphate kinase B (NDP kinase B; | 0,4 |
| NDK B) | |
| Glutathione S-transferase Pi 1 (GSTPIB) | 0,4 |
| adenylate cyclase 6 | 0,4 |
| D-Binding Protein (DBP) | 0,3 |
| transcription factor S-II | 0,3 |
| prostaglandin D2 synthase (21 kDa, brain) | 0,2 |

The use of a conditional inducible model such as has been described should permit the identification of the molecular determinants implicated in cardiopathy. The remodelling of the extracellular matrix and the cardiac functional alterations are in fact dynamic reversible processes when the signalling through mMR is restored.

The analysis of the differential expression of genes at different stages of the phenotypic reversion should in particular lead to the identification of the signalling routes implicated in the establishment and then the reversion of the cardiac fibrosis, thus opening up novel therapeutic approaches in the cardiovascular domain.

### BIBLIOGRAPHIC REFERENCES

Abremski et al, (1983), Studies of the properties of P1 site-specific recombination: evidence for topologically unlinked products following recombination, Cell, 32:1301-1311

Baron et al, (1995) Co-regulation of two gene activities by tetracycline, via a bidirectional promoter, Nucleic Acids Res. 17:3605-3606

Brocard et al, A chimeric Cre recombinase inducible by synthetic,but not by natural ligands of the glucocorticoid receptor. Nucleic Acids Res. Sep. 1, 1998;26(17):4086-90).

Berger et al, (1998). Mineralocorticoid receptor knockout mice: Pathophysiology of Na⁺ metabolism. Proc. Natl. Acad. Sci. USA, 95, 9424-9429

Choulika et al, (1994). The yeast I-Sce I meganuclease induces site-directed chromosomal recombination in mammalian cells. C. R. Acad. Sci. Paris 317, 1013-9.

Cohen-Tannoudji et al, (1998). I-SceI-induced gene replacement at a natural locus in embryonic stem cells. Molecular & Cellular Biology 18, 1444-8.

Dutt et al., (2000) Proteomic analysis. Curr Opin Biotechnol 2, 176-179.

Furth et al, (1994). Temporal control of gene expression in transgenic mice by a tetracycline-responsive promoter. Proceedings of the National Academy of Sciences of the United States of America 91, 9302-6.

Gossen et al, (1992). Tight control of gene expression in mammalian cells by tetracycline-responsive promoters. Proceedings of the National Academy of Sciences of the United States of America 89, 5547-51.

Gossen, et al, (1995). Transcriptional activation by tetracyclines in mammalian cells. Science 268, 1766-9.

Kistner et al, (1996). Doxycycline-mediated quantitative and tissue-specific control of gene expression in transgenic mice. Proceedings of the National Academy of Sciences of the United States of America 93, 10933-8.

Le Menuet et al, 2000. Transgenic mouse models to study human mineralcorticoid receptor function in vivo. Kidney International, vol. 57, 1299-1306.

Mansuy et al, (1998a). Restricted and regulated overexpression reveals calcineurin as a key component in the transition from short-term to long-term memory. Cell 92, 39-49.

Mansuy et al, (1998b). Inducible and reversible gene expression with the rtTA system for the study of memory. Neuron 21, 257-65.

Mayford et al, (1996). Control of memory formation through regulated expression of a CaMKII transgene. Science 274, 1678-83.

Hogan, B., Beddington, R., Costantini, F. and Lacy, E. (1994). Manipulating the mouse embryo. Plainview, N.Y.: Cold Spring Harbor Laboratory Press.

Indra et al, Temporally-controlled site-specific mutagenesis in the basal layer of the epidermis: comparison of the recombinase activity of the tamoxifene-inducible Cre-ER(T) and Cre-ER(T2) recombinases. Nucleic Acids Res. Nov 15, 1999;27(22):4324-7)

Kellendonk et al, Inducible site-specific recombination in the brain. J Mol Biol. Jan. 8, 1999;285(1):175-82).

Logie et al, (1995) Ligand-regulated site-specific recombination, Proc. Natl. Acad. Sci. USA 92(13):5940-5944

Metzger et al, (1995), Proc. Natl. Acad. Sci., Conditional site-specific recombination in mammalian cells using a ligand-dependant chimeric Cre recombinase, 92:6991-6995

Metzger, D. and Feil, R. (1999). Engineering the mouse genome by site-specific recombination. Current Opinion in Biotechnology 10, 470-6.

No et al, (1996). Ecdysone-inducible gene expression in mammalian cells and transgenic mice. Proc. natl. Sci. USA 93, 3346-3351.

Pitt et al, (1999), The effect of spironolactone of morbidity and mortality in patients with severe heart failure, the New England Journal of medecine, 341:709-717.

Robert et al, (1995), Biological determinants of Aldosterone-induced cardiac fibrosis in rats. Hypertension, 26(1):971-978

Sambrook et al, (1989) Molecular cloning, a laboratory Manual, Cold Spring Harbor Laboratory Press

Schena et al., (1998) Microarrays: biotechnology's discovery platform for functionnal genomics. Trends Biotechnol 16(7), 301-306.

Wang et al, (1997). Ligand-inducible and liver-specific target gene expression in transgenic mice. Nature Biotechnology 15, 239-243.

Yu et al, (1996), Conditional transgene expresion in the heart. Circ. Res. October 79(4):691-7

1. Nucleic acid which codes for the murine mineralocorticoid receptor, comprising the sequence SEQ ID no. 1 and/or no. 2.

2. Nucleic acid which blocks the expression of the murine mineralocorticoid receptor, the said receptor being coded by the nucleic acid as claimed in claim 1.

3. Nucleic acid as claimed in claim 2, consisting of a sequence of approximately 50 to 2000 bases complementary to the sequence SEQ ID no. 1 or no. 2, starting from the nucleotide no. 1 of SEQ ID no. 1.

4. Nucleic acid as claimed in claim 3, consisting of the sequence of 316 base pairs (bp) complementary to the sequence SEQ ID no. 1.

5. Nucleic acid construct comprising a nucleic acid as claimed in one of claims 2 to 4, of which the sequence is designated “antisense sequence”, in association with elements permitting the expression of the said antisense sequence.

6. Construct as claimed in claim 5, also comprising one or more elements which render the expression of the said antisense sequence inducible or conditional.

7. Construct as claimed in claim 5, in which the antisense sequence is associated with an operating sequence of the system of resistance to tetracycline of the bacterial transposon Tn10.

8. Host cell into which at least one construct as defined in one of claims 5 to 7 has been transferred in a stable manner.

9. Host cell as claimed in claim 8, characterised in that it is a mouse ovocyte.

10. Non-human transgenic animal, preferably a mouse, in which at least one nucleic acid construct as claimed in one of claims 5 to 7 is integrated in the genome.

11. Method of obtaining a non-human transgenic animal, preferably a mouse, in which the expression of the mineralocorticoid receptor is suppressed in an inducible or conditional manner, comprising a step of integrating into the genome of a non-human animal a nucleic acid construct comprising a nucleic acid as claimed in one of claims 2 to 4, of which the sequence is designated as an “antisense sequence”, and a step of crossing the non-human transgenic animal thus obtained with a non-human transgenic animal in which a nucleic acid construct carrying elements permitting the regulation of the expression of the said antisense sequence in an inducible or conditional manner is integrated into the genome.

12. Method as claimed in claim 11, in which the said elements permitting the regulation of the expression of the said antisense sequence are specific to a tissue of the animal.

13. Non-human transgenic animal, preferably a mouse, capable of being obtained by the method as claimed in claim 11 or 12.

14. Non-human animal as claimed in claim 13, in which the expression of the mineralocorticoid receptor is suppressed in the heart.

15. Use of a non-human animal as claimed in one of claims 10, 13 or 14 for the screening of therapeutic agents effective in the prevention and/or the treatment of human or animal disorders or pathologies in which the mineralocorticoid receptor is implicated.

16. Use as claimed in claim 15, for the screening of therapeutic agents effective in the prevention and/or the treatment of a cardiac fibrosis and/or a cardiac insufficiency.

17. Use of a non-human animal model as claimed in one of claims 10, 13 or 14 for the identification of genes or proteins having a differential expression over the course of human or animal pathologies in which the mineralocorticoid receptor is implicated.

18. Cells isolated and possibly cultivated from the non-human animals as claimed in one of claims 10, 13 or 14.

19. Use of the cells as claimed in claim 18 for the screening of therapeutic agents effective in the prevention and/or the treatment of human or animal disorders or pathologies in which the mineralocorticoid receptor is implicated, or for testing the toxicity of molecules with therapeutic potential.

20. Use of the cells as claimed in claim 18 for the identification of genes or proteins having a differential expression over the course of human or animal pathologies in which the mineralocorticoid receptor is implicated.


SEQUENCE LISTING (6 sequences)

SEQ ID NO: 1 - DNA, 316 bp, Mus musculus
ggctaccacagtctccctgaaggcctagatatggaaaggcgctggagtcaagtgtctcagaccttggagcgttcttctcttggacctgcagagaggaccaatgagaacagctacatggagattgtcaacgtcagctgcgtttccggtgctactccgaacaacagtactcaagggagcagcaaagaaaaacacgaattactcccttgtcttcagcaagacaatagtcggtctgggattttgccatcagatattaaaactgagctggaatccaaggaactttcagccacggtggctcagtccatggatttatatatgg

SEQ ID NO: 2 - DNA, 320 bp, Mus musculus
aagagccctatcatctgtcatgagaagagcccctctgtttgcagcccgctcaacatgccgtcttcagtatgcagccccgcgggcatcaactccatgtcctcctccacagctagctttggcagtttcccagtgcacagtcccatcactcaaggaacctcactgacatgctcccccagtgttgaaaatagaggctcaaggtcacacagccccgtacatgcgagcaatgtgggctctcctctttcaagtccattaagcagcatgaaatccccaatttccagccctccaagtcactgcagtgtaaaatctccagtctccagtcc

SEQ ID NO: 3 - DNA, 23 bp, Mus musculus
ggctaccacagtctccctgaagg

SEQ ID NO: 4 - DNA, 22 bp, Mus musculus
ccatatataaacccatggactg

SEQ ID NO: 5 - DNA, 26 bp, Mus musculus
aagagccctatcatctgtcatgagaa

SEQ ID NO: 6 - DNA, 28 bp, Mus musculus
ggactggagactggagattttacactgc